(19) United States
(12) Patent Application Publication
(10) Pub. No.: US 2020/0234819 A1
(43) Pub. Date: Jul. 23, 2020

(54) SYSTEM AND METHOD FOR COORDINATION OF SURGICAL PROCEDURES

(71) Applicant: Time Out Surgical, LLC, New Palestine, IN (US)

(72) Inventor: Nickolas H. Keller, New Palestine, IN (US)

(21) Appl. No.: 16/751,125

(22) Filed: Jan. 23, 2020

Related U.S. Application Data
(60) Provisional application No. 62/795,933, filed on Jan. 23, 2019.

(51) Int. Cl. G16H 40/20

(52) U.S. Cl. CPC G16H 40/20; G06K 7/10297

(57) ABSTRACT

A method for tracking medical procedure data among entities includes receiving and storing a new case entry and corresponding new case data. The method further includes providing a new case notification to at least a facility device and a representative device. Additionally, the method includes receiving a request for a medical device and storing the request, and communicating the request to the representative device. The method includes receiving a recommended medical device associated with the request and storing the recommended medical device.

### CROSS-REFERENCE TO RELATED APPLICATIONS

This application claims the benefit of U.S. Provisional Patent Application Ser. No. 62/795,933 filed on Jan. 23, 2019 and entitled “System and Method for Coordination of Surgical Procedures,” which is herein incorporated by reference in its entirety.

### BACKGROUND OF THE INVENTION

The subject matter disclosed herein relates generally to electronic medical procedure coordination systems and methods, and, more particularly, to computer-implemented systems and methods that provide a platform that interfaces with the entities involved in a medical device procedure to coordinate device selection (e.g., implants, instruments), procedure scheduling, sterilization timing, and other phases of the procedure.

The complete process of using and/or implanting a medical device, from the initial selection of devices and materials to surgical use of the implant and/or instruments, can require extreme coordination and communication among the entities involved. In a basic case, the surgical process involves three entities: the medical device (“device”) manufacturer representative (“rep”), the hospital where the procedure takes place (“facility”), and the doctor performing the surgery (“surgeon”).

FIG. 1 illustrates a conventional process 10 in which entities involved in a medical device procedure exchange data about the procedure in order to coordinate the logistics (e.g., time, location, required devices, etc.). As mentioned above, this process often relies on communicating both efficiently and effectively.

As shown, a surgeon 12 (or their “scheduler”/assistant) must communicate with both a manufacturer representative 16 and facility coordinator(s) 14. Similarly, the manufacturer rep 16 may communicate with both the facility coordinator(s) 14 and/or the surgeon 12. The communication processes 18, 20, 22 may occur via several rounds of phone calls, emails, or the like. Further, the communication processes 18, 20, 22 can occur across several devices (e.g., smartphones, tablets, desktop computers, landline telephones, etc.). Understandably, accidental delays in returning messages from any of the three entities can sometimes lead to last minute concerns. The conventional process 10 can be heavily dependent on the responsiveness of each entity. Navigating this process for each procedure, and with each new rep and/or facility can vary for the surgeon 12 (or their scheduler/assistant).

Within conventional processes (such as process 10), there is no closed-loop communication system for when a surgeon schedules a procedure (i.e., surgery). The surgeon's scheduler may call the hospital/facility and either read off the device needed, procedure description, patient information, time, etc., or the scheduler can email that information to the facility. Next, a member of the facility staff may have to call each rep (a single surgeon can potentially have cases in one morning with eight reps, from eight different manufacturers) and perform the same information transfer.

Conventional processes often depend on personal preferences. Some schedulers text the rep, some call and leave voicemails, some email all the upcoming cases out on a certain day of the week. Everybody has a different “system” for relaying this information.

At some hospitals/facilities, a designated point-person reaches out to the reps to make the hardware/implant/device request, sometimes with encrypted emails. Other facilities do not have a designated point-person, and/or require more context when the information is provided. Some facilities don't make the device request with the reps, and instead have the surgeon's office handle the request.

Schedulers must generally be detailed-oriented and organized. This helps avoid any day-of-surgery requests. At that point, there is little that the rep can do, because devices that have to be sterilized need to be to the facility twenty-four hours prior to the surgery for processing.

When the communication process between the entities is not conducted flawlessly, the surgeon may not have the desired device(s) in time for the surgery. In these situations, the surgeon may now be forced to use the older, first-generation technology that the hospital bought decades ago.

If the facility is a busy surgery center or hospital, and the older, first generation technology was needed for another recent surgery, then there may not be another alternative available. In this case, the surgeon has to reschedule the patient, which can be very expensive for the surgery center (as well as inconveniencing the patient). If rescheduling the surgery is not feasible, the facility has to “flash” sterilize or autoclave the device, which can put the facility at serious liability risk. Flash sterilization is often regarded as not being as thorough as doing the full multi-hour process. Accordingly, standard of care is to never “flash” plates, screws, instruments, or metal devices and implants. Facilities will only do this in an extreme emergency, and the surgeon must sign off on the decision, and explain why it is medically necessary.

In conventional systems, the above process is at least partially executed on paper, with forms being completed and reviewed manually. Additionally, those steps that include some automation and/or digitization of information produce electronic records, such as spreadsheets, electronic documents, emails with attachments, etc., are neither substantively harmonized nor made easy to access according to a common operating procedure.

Coordination among entities can be difficult, as one surgeon may work with many device manufacturers (each having multiple reps), with many device types, and at several different facilities. Coordination issues can result in missing devices at surgery time, or flash sterilization of devices, among other timing concerns. Existing scheduling systems have been unsuccessful at overcoming these issues to enable efficient and verified surgery scheduling that eases the communication burden for each entity. It would be desirable to provide systems and methods that implement a coordination platform for device procedures that can be used by entities acting in a procedure, to increase accuracy and reduce inefficiencies.

### SUMMARY

One aspect of the present disclosure is a system. The system includes one or more hardware computing devices including a processor and memory storing program instructions that, when executed by the processor, cause the one or more hardware computing devices to provide, to a plurality of user computing devices in electronic communication with the one or more hardware computing devices over a network, a medical device coordination platform. The medical device coordination platform is configured to receive first user input data from a first device of a plurality of user devices, the first device associated with a medical provider. The medical device coordination platform further receives second user input data from a second device of the plurality of user devices, the second device associated with a representative of a medical device provider. Further, the medical device coordination platform receives third user input data from a third device of the plurality of user devices, the third device associated with a medical facility. At least one of the first, second, and third user input data corresponds to a preparation status. The medical device coordination platform is configured to generate and send a message to at least one of the plurality of user devices in response to the preparation status corresponding to a predetermined status.

Another aspect of the present disclosure is a method for tracking medical procedure data among entities. The method includes receiving and storing a new case entry and corresponding new case data. The method further includes providing a new case notification to at least a facility device and a representative device. Additionally, the method includes receiving a request for a medical device and storing the request, and communicating the request to the representative device. The method includes receiving a recommended medical device associated with the request and storing the recommended medical device.

### BRIEF DESCRIPTION OF THE DRAWINGS

The present invention will become more apparent from the detailed description set forth below when taken in conjunction with the drawings, in which like elements bear like reference numerals.

FIG. 1 is a block diagram of an existing manual system and method for coordinating surgical procedure data among entities;

FIG. 2 is a block diagram of a coordination platform for streamlining communication among relevant surgical entities, in accordance with the present disclosure;

FIG. 3 is a detailed block diagram of the coordination platform of FIG. 2, in accordance with the present disclosure;

FIG. 4 is a block diagram of exemplary inputs and outputs corresponding to the coordination platform of FIG. 2, in accordance with the present disclosure;

FIG. 5A is an example user device for displaying a graphical user interface (GUI), in accordance with the present disclosure;

FIG. 5B is another example user device for displaying a GUI, in accordance with the present disclosure;

FIG. 6A is an example GUI for coordinating procedures, in accordance with the present disclosure.

FIG. 6B is another view of the example GUI of FIG. 6A, in accordance with the present disclosure; and

FIG. 6C is another view of the example GUI of FIG. 6A, in accordance with the present disclosure.

FIG. 7 is an example process diagram for editing a user profile, in accordance with the present disclosure.

FIG. 8 is an example process diagram for case management, in accordance with the present disclosure.

FIG. 9A is an example screen corresponding to a vendor view, in accordance with the present disclosure.

FIG. 9B is an example screen corresponding to a facility view, in accordance with the present disclosure.

FIG. 10A is a portion of an example process diagram for creating a case, in accordance with the present disclosure.

FIG. 10B is another portion of the example process diagram of FIG. 10A, in accordance with the present disclosure.

FIG. 11 is an example process diagram for creating a case, in accordance with the present disclosure.

FIG. 12 is an example process diagram for reviewing a case, in accordance with the present disclosure.

FIG. 13 is an example process diagram for sharing a case, in accordance with the present disclosure.

FIG. 14 is an example process diagram for tracking delivery and check in, in accordance with the present disclosure.

FIG. 15 is an example process diagram for tracking a representative, in accordance with the present disclosure.

FIG. 16 is an example process diagram for viewing cases, in accordance with the present disclosure.

### DETAILED DESCRIPTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures. The figures depict selected embodiments and are not intended to limit the scope of embodiments of the invention. Skilled artisans will recognize the examples provided herein have many useful alternatives and fall within the scope of embodiments of the invention.

The invention may be described herein in terms of functional and/or logical block components and various processing steps. It should be appreciated that such block components may be realized by any number of hardware, software, and/or firmware components configured to perform the specified functions. For example, an embodiment may employ various integrated circuit components, e.g., memory elements, digital signal processing elements, logic elements, diodes, look-up tables, etc., which may carry out a variety of functions under the control of one or more microprocessors or other control devices. Other embodiments may employ program code, or code in combination with other circuit components.

In accordance with the practices of persons skilled in the art of computer programming, the present disclosure may be described herein with reference to symbolic representations of operations that may be performed by various computing components, modules, or devices. Such operations may be referred to as being computer-executed, computerized, software-implemented, or computer-implemented. It will be appreciated that operations that can be symbolically represented include the manipulation by the various microprocessor devices of electrical signals representing data bits at memory locations in the system memory, as well as other processing of signals. The memory locations where data bits are maintained are physical locations that have particular electrical, magnetic, optical, or organic properties corresponding to the data bits.

As non-limiting examples unless specifically indicated, any database or data store described herein may comprise a local database, online database, desktop database, server-side database, relational database, hierarchical database, network database, object database, object-relational database, associative database, concept-oriented database, entity-attribute-value database, multi-dimensional database, semi-structured database, star schema database, XML database, file, collection of files, spreadsheet, or other means of data storage located on a computer, client, server, or any other storage device known in the art or developed in the future. File systems for file or database storage may be any file system, including without limitation disk or shared disk, flash, tape, database, transactional, and network file systems, using UNIX, Linux, Mac OS X, Windows FAT or NTFS, FreeBSD, or any other operating system.

The various aspects of the disclosure will be described in connection with providing a coordination platform that enables entities involved in a medical device procedure to digitize, reconcile, and otherwise manage information related to the device procedure. That is because the features and advantages that arise due to the invention are well suited to this purpose and overcome one or more of the drawbacks of previous systems for addressing the same problem of surgical procedure data management. However, it should be appreciated that the disclosure is applicable to other procedures and to achieve other objectives as well.

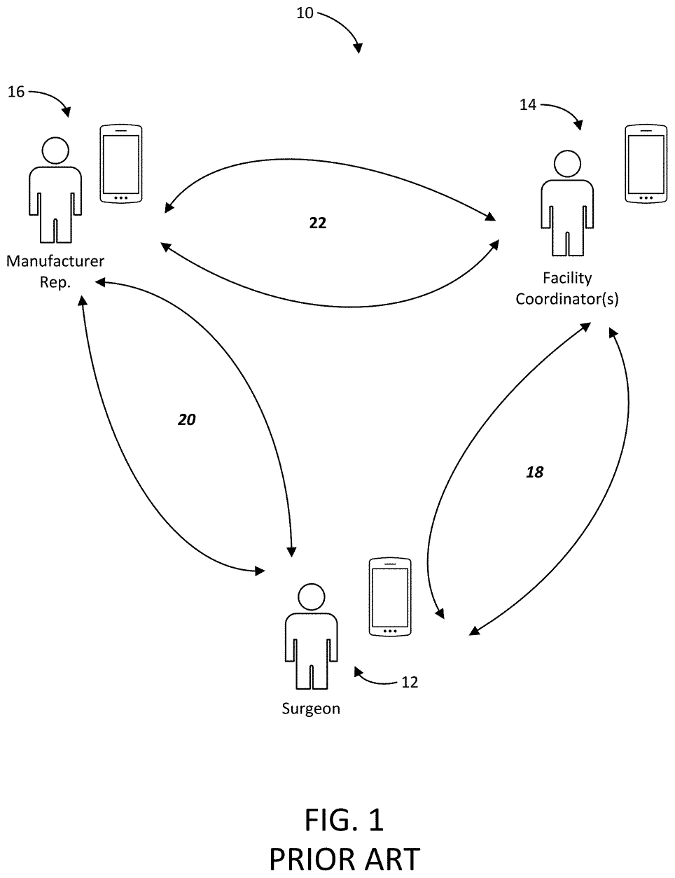

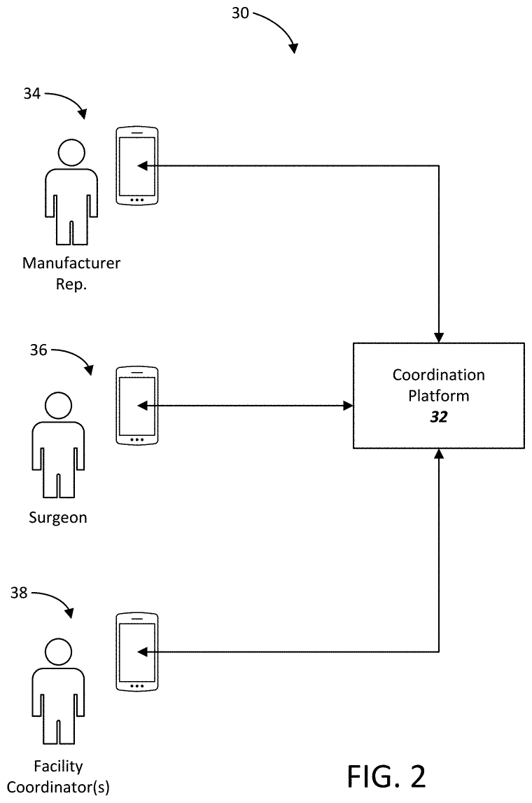

FIG. 2 is a block diagram of a coordination platform 32 for streamlining communications 30 among relevant surgical entities, in accordance with the present disclosure. As shown, the entities involved in a medical device procedure may use their computing devices (e.g., smartphones) to access a coordination platform 32 that enables the entities to electronically interact with one another to determine the devices and scheduling for a surgery. Notably, each of the manufacturer representative 34 (“rep”), the surgeon 36, and the facility coordinator(s) 38 need only to communicate with the coordination platform 32. This is distinct from conventional scheduling processes, such as the process 10 described with respect to FIG. 1, above. The coordination platform 32 may send and receive data to computing devices in real-time, enabling the rep 34, surgeon 36, and facility coordinator(s) 38 to both monitor and update surgery information as-needed.

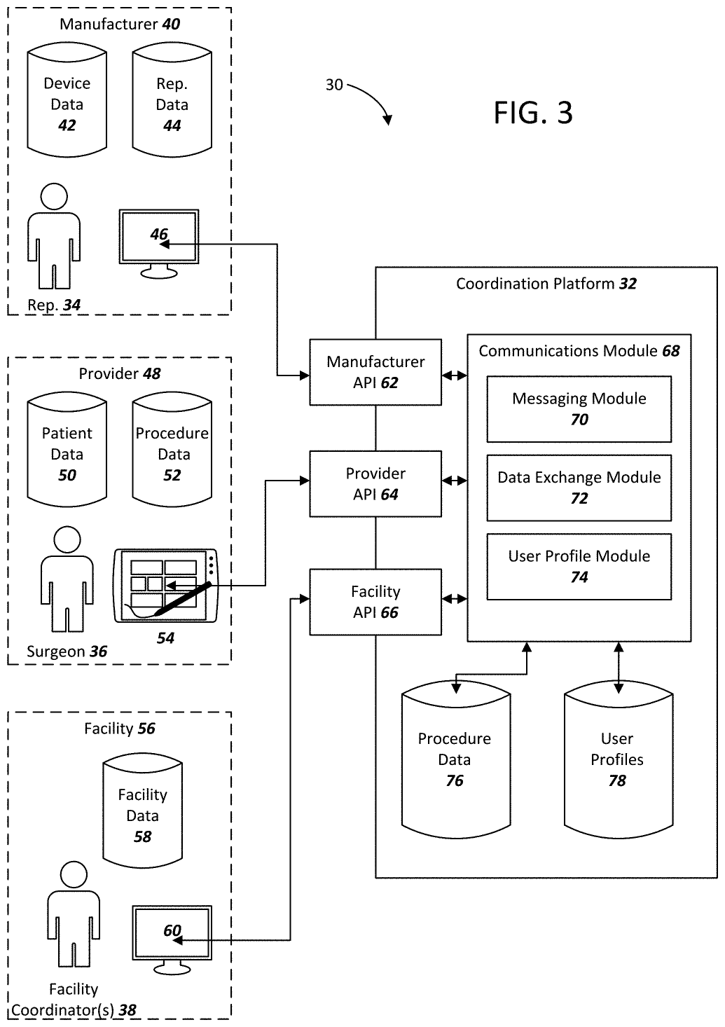

FIG. 3 is a detailed block diagram of the coordination platform 32 for communications 30 among relevant surgical entities, in accordance with the present disclosure. The coordination platform 32 may include and/or be implemented using computing hardware and software that provides computational resources of at least one physical computing device to one or more user devices that interface with the platform 32 to execute services and store and access data. The data may include data related to a particular surgical procedure, and the various modules (68-74) may be used to coordinate the data between entities. The platform 32 may be implemented in the cloud (on one or more virtual machines), on a remote server, or across multiple servers communicating with each other over an electronic network. Additionally or alternatively, the platform 32, or parts thereof, may be implemented on one or more user devices (see, e.g., user devices 46, 54, and 60), such as a mobile phone, tablet, or computer, using the hardware and software interfaces of the mobile device, including communicating with a user of the mobile device via sound or visual displays, and communicating with objects on remote devices, such as data stores of the platform 32, via the internet or a cellular network. For example, a user of a mobile device may install on the mobile device an application, software service, firmware, or other device client that serves as the interface to the platform 32 or otherwise accesses the platform 32 modules.

The entities described above with respect to FIG. 2 may be enabled to use the platform 32 and exchange data with the other entities as described herein. In comparison to existing systems that use a combination of manual and uncoordinated electronic processes, the platform 32 and associated systems provide significantly improved efficiency, accuracy, and simplicity in entering, validating, resolving, and processing data from the multiple entities in order to complete scheduling needs for medical device surgeries.

The rep 34 may access the platform 32 using one or more user devices 46 that store or connect to an application programming interface (API) 62 that provides access to the platform 32. The user device 46 can send data, such as medical device and product data stored in one or more local data stores 42, to the platform 32 for processing, and can send data (e.g., messages) through the platform 32 to other entities, as described further below. Additionally, the user device 46 can send data, such as rep availability and location stored in one or more local data stores 44, to the platform 32 for processing.

Similarly, user device 54 corresponding to provider 48 (e.g., surgeon's office or clinic) may provide appropriate patient data, as stored within one or more local data stores 50, to the coordination platform 32 via API 64. Further, the user device 54 can send data, such as required equipment/staffing and surgeon availability stored in one or more local data stored 52, to the platform 32 for processing.

Also similarly, user device 60 corresponding to the facility 56 (e.g., hospital, surgery center) may use a facility API 66 to access the platform 32 and use the platform modules to manage data, such as facility hours, room availability, equipment availability, etc., as stored in one or more local data stores 58. As described above, mobile devices may also be used with the platform 32, thus, mobile users such as the surgeon 36 and rep 34 may use their respective mobile devices (not shown) to access the platform 32, such as through device clients installed on the respective mobile devices.

Any of the APIs 62-66, and other applications and user interfaces for accessing the platform 32 may be implemented in any suitable software framework, such as MICROSOFT .NET, Amazon Web Services, ActiveX, SOAP, and the like. In some embodiments, each API may be embodied in a web application that is operable in, and/or accessible by, an internet browser installed on the various user devices 46, 54, 60. The web application may provide a graphical user interface using any suitable internet technology, such as HTML. In some embodiments, all messages and other data exchanges pertaining to data that is managed by the platform 32, including messages between entities, may be passed through the platform 32 via the corresponding APIs and device clients, which may perform one or more of input validation, error handling, and interfacing with particular platform 32 modules. Facilitating inter-entity and entity-to-service messaging through the APIs can allow for standardization of message formatting and data access.

The platform 32 may implement or support a communications module 68 that coordinates invocations of various services of the platform 32. Such coordination may include acquiring and managing computing resources of the physical computing devices, providing interfaces to local and/or remote data stores, receiving, formatting, parsing, processing, and delivering requests between requesting and target devices, and the like. FIG. 3 illustrates some exemplary modules that can be made accessible to user devices according to preset permissions, such as those corresponding to an authentication scheme. For example, the user profile module 74 may include or communicate with an authorization system that checks credentials (e.g., username/password combination, secure socket layer certificate, private key, etc.) presented by a user device, and the user profile module 74 may grant certain access to certain services and to certain data stores according to the user authentication/authorization. Further, the user profile module 74 may receive data submitted by a user device in order to generate and/or maintain a user profile associated with an entity using the system. The user profile module 74 may, for example, store this data in a user profiles store 78 in accordance with any suitable data structure or data record configuration. Various entities may have one or more user profiles associated therewith, such as a manufacturer 40, one or more particular users associated with the manufacturer 40 (e.g., various reps, rep 34), a medical facility 56, employees or departments of the medical facility 56 (e.g., schedulers, assistants), and a surgeon 36. A user profile may have various permissions associated therewith, such as restrictions on which services can be used and which data stores can be accessed. Further, in some embodiments, user preferences may be stored within user profiles store 78. For example, the surgeon 36 may prefer a certain manufacturer for one device type. Accordingly, this manufacturer may be stored as the default for that procedure type and that specific surgeon. In various embodiments, the user profile of an entity includes information that can be used by other modules to coordinate data related to one, some, or all implant/device procedures with the relevant entities. Some types of such information may be common to all entities and to all user profiles, such as identifying information (e.g., name and address of hospital), contact information, and the like.

A data exchange module 72 may coordinate the exchange of information related to a surgical procedure between entities using the platform 32. The module 72 and other modules of the communications module 68 may begin administration of a particular surgical procedure upon requested scheduling of the procedure. In other embodiments, the new surgical scheduling may be initiated upon receipt by the data exchange module 72 of one or more orders for devices and/or products for use in the surgical procedure.

Data related to processing a particular surgical procedure may be stored in various data stores for different uses. For example, the data can be stored in a procedure data store 76 that aggregates data for particular surgical procedures and/or for certain devices and products, in order to refine common parameters used by the system. For example, the data can be used to determine that a certain number of units of an ancillary product are typically used in a particular surgical procedure (e.g., specialized screws).

A messaging module 70 may facilitate communications between the different entities using the system. The messaging module 70 may be configured to convert between different formats of messages, such as when sending messages from a mobile device to a desktop or web application. The messaging module 70 may also identify usage permissions of different users attempting to send messages, to determine whether the users are authorized to do so. Messages, and any other data, may be encrypted and/or sterilized in order to satisfy any applicable data security regulations, particularly with respect to health care and personally-identifying information.

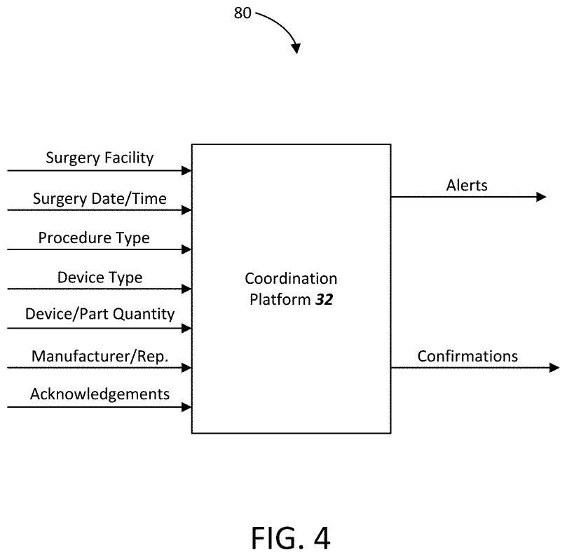

FIG. 4 is a block diagram 80 of exemplary inputs and outputs corresponding to the coordination platform of FIG. 2, according to some embodiments. As shown, the coordination platform 32 may receive inputs from the various entities (e.g., manufacturer 40/rep 34, provider 48/surgeon 36, facility 56/facility coordinator(s) 38), including, but not limited to: the desired surgery facility, the surgery date/time, the type of procedure, the desired device type, the desired quantity corresponding to the device/part, the requested manufacturer/rep, and acknowledgements.

A key issue with conventional scheduling systems is not knowing, with certainty, that each entity has completed preparation prior to the surgery time. Accordingly, the present disclosure provides “acknowledgement” features via a scheduling graphical user interface, which is discussed in detail below. Key entities and individuals involved with procedure and medical device scheduling may be required to affirm that their portion of surgery preparation is complete. As one example, the rep 34 may affirm, via the coordination platform 32, that they have received the device request and/or that the device has been timely delivered to the facility.

Still referring to FIG. 4, the coordination platform 32 may provide several outputs to the various entities (e.g., via user devices). As shown, the coordination platform 32 may provide alerts and/or confirmations. As one example, an alert may be sent to each entity, via messaging, if portions of the surgery preparation are marked incomplete after a specified time (e.g., twenty-four hours before the scheduled surgery time). A message may also be sent based on a user request (e.g., the surgeon wants quick verification from a rep). Additionally, coordination platform 32 may send confirmations to each entity, via messaging, once all portions of the surgery preparation are marked complete. By providing real-time feedback from each entity, last minute scheduling concerns (e.g., having to “flash” the device) can be avoided.

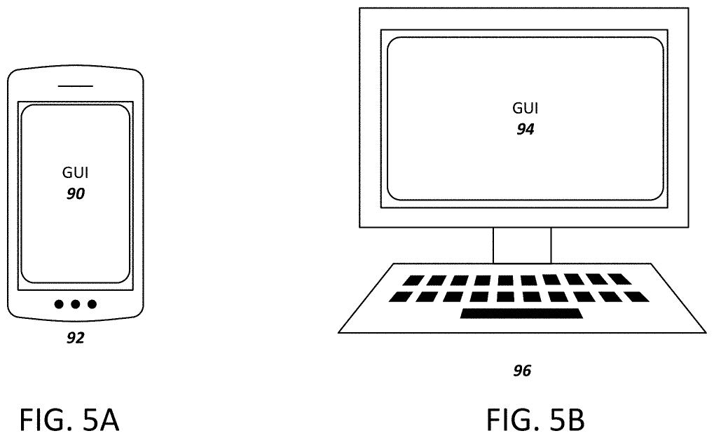

Referring now to FIGS. 5A and 5B, two example graphical user interfaces 90, 94 (GUIs) are shown. In some embodiments, one or more of the entities discussed above may use a mobile device (such as a smart phone 92). Similarly, one of more of the entities above may use a desktop computer 96 or other internet-configured device. The smart phone 92 may be configured to locate and download an application corresponding to the coordination platform 32. The application may enable the user to send and receive scheduling data to/from the coordination platform 32. Additionally, the application can provide GUI 90, which can be generally formatted for display on a mobile device such as smart phone 92. GUI 90 can accept user inputs and display platform outputs.

The desktop computer 96, or other internet-configured devices, may be configured to access a website (via an internet browser) corresponding to the coordination platform 32. The website may enable the user to send and receive scheduling data to/from the coordination platform 32. Additionally, the website can provide GUI 94, which can be generally formatted for display on a computer monitor. GUI 94 can accept user inputs and display platform outputs.

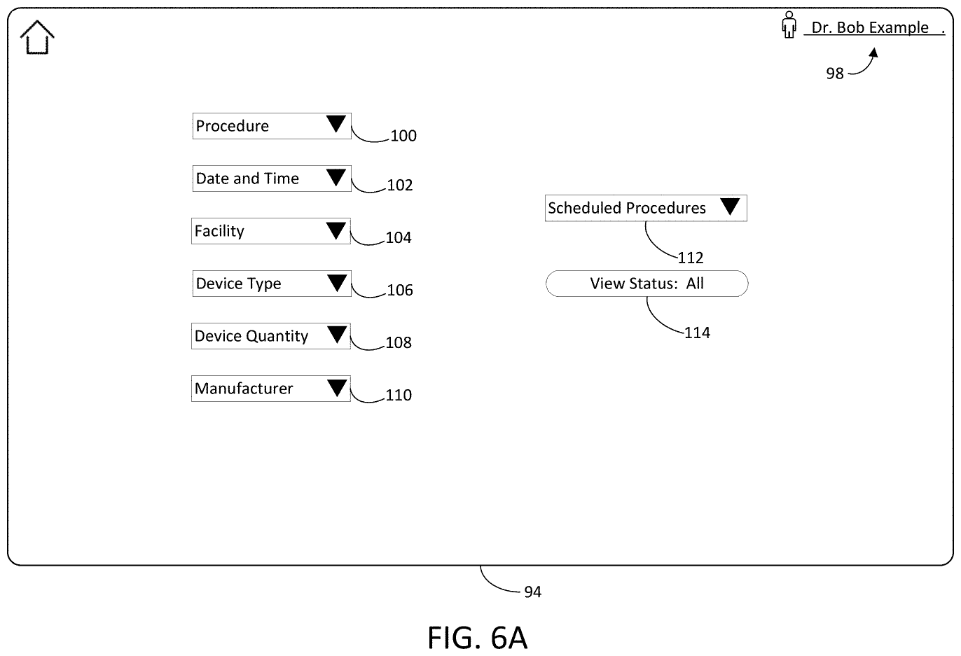

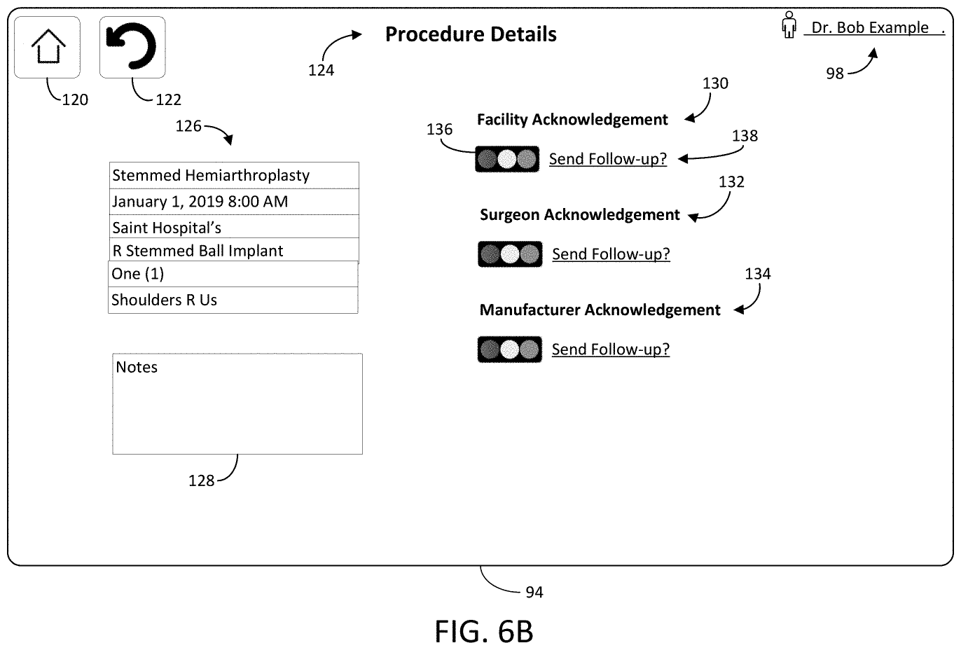

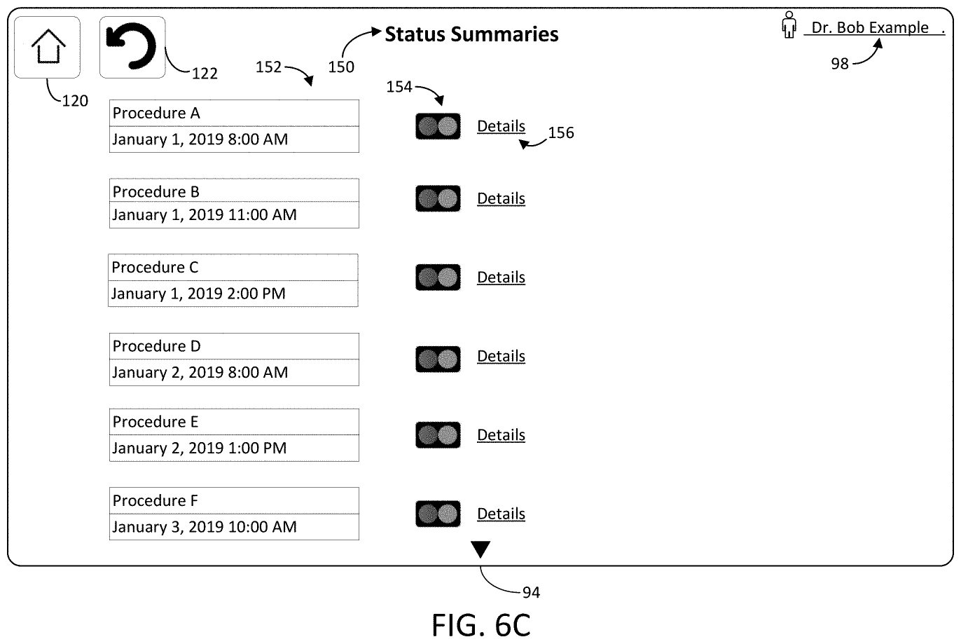

Referring now to FIGS. 6A-6C, the GUI 94 for coordinating procedures is shown, according to some embodiments of the present disclosure. In some embodiments, GUI 90 may function the same or similar to GUI 94. In some embodiments, GUI 90 may be similar to GUI 94, but resized to accommodate the display size of the smart phone 92.

As shown, a homepage of GUI 94 can include a user ID 98, which can be updated based on which user is logged in to the coordination platform 32. Here, for example, a surgeon is currently logged in. The GUI 94 can include several drop-down menus, which are configured for the surgeon to select desired procedure attributes. The drop-down menus can be used to book a new procedure, for example. As shown, a procedure menu 100 can provide a selectable list of procedure types, a date and time menu 102 can provide a selectable calendar and/or list of procedure times, a facility menu 104 can provide a selectable list of available facilities, a device type menu 106 can provide a selectable list of devices, a device quantity menu 108 can provide a selectable list of quantities, and a manufacturer menu 110 can provide a selectable list of manufacturers. Additional drop-down menus and/or fillable fields may be provided via GUI 94.

In some embodiments, the options displayed within each drop-down menu may be updated based on other, currently selected options. As an example, when the device type is selected via menu 106, the list of manufacturers included in menu 110 may be updated to include only those that can supply the desired device type. Notably, menus 100-110 may not be included on the homepage for reps and/or facility coordinator(s), as they typically do not book the procedures at the initial booking.

As described above, default options may auto-populate drop-down selections based on user profile settings. As one example, if a surgeon prefers a particular manufacturer for certain devices, then the manufacturer may be selected by default once the surgeon selects a device (but can be manually changed). In some embodiments, the facility selection can be auto-filled based on a current location of the user (e.g., the surgeon). The desktop computer 96 can, for example, provide an IP address to the coordination platform 32, which can subsequently determine if the surgeon is currently within a known facility. Similarly, the smart phone 92 can, for example, provide location information to the coordination platform 32 (e.g., via GPS), which can subsequently determine if the surgeon is currently within a known facility. Accordingly, the facility selection can default to the surgeon's current facility.

As shown, the home page, as displayed via GUI 94 can include a drop-down menu 112 in order to provide a selectable list of scheduled procedures. Upon selection of a procedure from the list, GUI 94 may display details related to that scheduled procedure. Additionally, selectable button 114 may be displayed via GUI 94. Upon selection of the button 114, an entity (e.g., the surgeon) may view status details for every currently scheduled procedure that they are involved with.

FIG. 6B is an example image of GUI 94, which can be displayed upon selection from menu 112 (scheduled procedures). As shown, a home button 120 can be displayed. Selection of the home button 120 can return the user to the display shown in FIG. 6A, for example. Similarly, a back button 122 can be displayed. Selection of the back button 122 can return the GUI 94 to the previous display.

The GUI 94 can display procedure details page 124. This may include a summary portion 126, which shows all the previously-selected procedure details. Additionally, a notes field 128 may be provided. The surgeon, facility coordinator(s), and/or the rep may enter text into the notes field 128. The GUI 94 can further display various acknowledgment sections, such as a facility acknowledgment 130, a surgeon acknowledgement 132, and/or a manufacturer acknowledgement 134. As described above, each entity may affirm when their portion of procedure preparation is complete. An indicator 136 may be provided, and can be configured to show a red indicator, a yellow indicator, and/or a green indicator. Alerts and/or confirmations can be sent to the entities involved in the specific procedure based on the status of each indicator 136. As one example, if the rep has not updated the manufacturer acknowledgment 134 to “green” twenty-four hours before the surgery, then an alert may be sent to one or more of the involved entities, e.g., to the surgeon or facility to contact the rep to follow up and/or to the rep itself as a reminder that confirmation or some other response is still needed. In some embodiments, the indicator 136 may default to red. In addition to the indicator 136, each acknowledgement section may include a selectable option 138 that corresponds to sending a follow-up to the corresponding entity. Follow up communications may be transmitted to the device of the user being contacted directly through the coordination platform 32. In that case, the coordination platform 32 may be configured to play an audible alert, provide a noticeable visual alert, e.g., causing the display screen of the user's device to flash repeatedly, and/or provide a vibratory or haptic alert to the user's device. Additionally or alternatively, the coordination platform 32 may send one or more of an e-mail, an SMS message, and an automated telephone call in order to get the user's attention. In this way, a surgeon, for example, can prompt a rep or facility coordinator to update the coordination platform 32.

The procedure details page 124 may include additional fields, text, and/or images. Further, portions of the procedure details page 124 may be displayed differently than what is described herein. As one example, the indicators 136 may be shown as selectable check-boxes, instead of selectable colors. In some embodiments, the procedure details page 124 may include an acknowledgment section specific to confirming that the device has arrived to the facility.

Referring now to FIG. 6C, an example image of the GUI 94 is shown, according to some embodiments. A status summaries page 150 may be displayed upon selection of selectable button 114 (see, e.g., FIG. 6A). The status summaries page 150 may include a procedures section 152. In some embodiments, the procedures section 152 may include summary details for each scheduled procedure (e.g., procedure type, date and time, etc.). In some embodiments, the status summaries page 150 may include an indicator 154 for each listed procedure in the procedures section 152. Additionally, a selectable details button 156 can be provided for each listed procedure. Selecting the details button 156 can update the GUI 94 to display a procedure details page 124.

In some embodiments, the indicator 154 can include a red indicator and a green indicator. The indicator 154 may change from red to green, once each entity (e.g., the rep, the surgeon, and the facility coordinator(s)) affirm that their portion of procedure preparation is complete (e.g., via procedure details page 124). Accordingly, the indicator 154 can provide a quick summary view for each procedure.

The status summaries page 150 may include additional fields, text, and/or images. Further, portions of the status summaries page 150 may be displayed differently than what is described herein. As one example, the indicators 154 may be shown as “Yes/No” statuses, instead of color indicators.

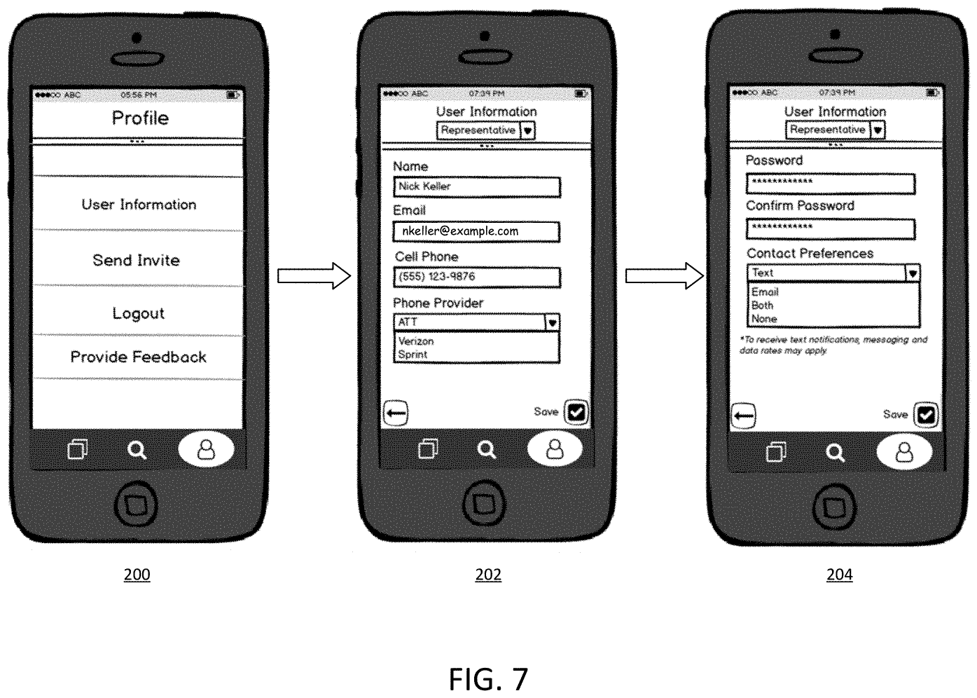

Referring now to FIGS. 7-16, various example screens corresponding to the platform 32 are shown, according to some embodiments of the present disclosure. The example screens shown in FIGS. 7-16 may be displayed via the GUI 94. As described above, the GUI 90 may function the same or similar to GUI 94.

In some embodiments, a user may be registered with the platform 32 via the GUI 94. As an example, a user may receive a registration invitation via an email, text message, or the like. In some embodiments, a login screen provided by GUI 94 can include an option for registering a new user. The registration process may be generally similar across all types of users (i.e., the rep, the surgeon, and the facility coordinator(s)). In some embodiments, the registration invitation may include instructions for accessing the platform 32, as well as a unique access code that may be temporarily associated with the user's known email or phone number. Upon accessing the platform 32, GUI 94 may prompt an unregistered user to input their email or phone, as well as the previously provided access code. The platform 32 may subsequently verify that the access code and email/phone match and are valid.

Referring now to FIG. 7, an example process diagram for editing a user profile is shown, in accordance with the present disclosure. A first user profile screen 200 may be provided via GUI 94. As shown, the first user profile screen 200 may be accessed by user selection of a “profile” icon within the GUI 94 menu (e.g., disposed at the bottom of each application screen). The first user profile screen 200 can include several options, such as “user information,” “send invite,” “logout,” and/or “provide feedback.” Upon selection of “user information,” a second user profile screen 202 may be provided via GUI 94. As shown, a user may input their information via various fields and/or dropdown menus (e.g., name, email, cell phone, phone provider). The second user profile screen 202 may also include a dropdown menu or other selection mechanism for inputting a “user type.” The user type options may include representative (“rep”), surgeon, and facility admin, according to some embodiments.

Still referring to FIG. 7, a third user profile screen 204 is shown. The third user profile screen 204 may be displayed via GUI 94, and may be accessed by scrolling from the second user profile screen 202, or via a new screen that is displayed once all fields are populated on the second user profile screen 202. The third user profile screen 204 may include various fields and dropdown menus corresponding to a user password, confirmation of the password, and/or contact preferences (e.g., text, email, both, none). Once a user is finished editing their profile via screens 200, 202, 204, they may select a “save” icon. The platform 32 then saves the user profile information (e.g., via user profiles database 78).

Depending on the selected “user type,” additional fields and/or dropdown menus may appear via screens 202, 204. As an example, if the user type is “surgeon,” the user may have the option to add one or more “delegates.” The user may input an email address corresponding to each desired delegate, which may provide the delegates with access to the platform 32. This function can be particularly useful, for example, if the surgeon wants to provide access to their cases to an assistant or surgical coordinator. As another example, if the user type is “facility admin,” the user may have the option to add a facility name.

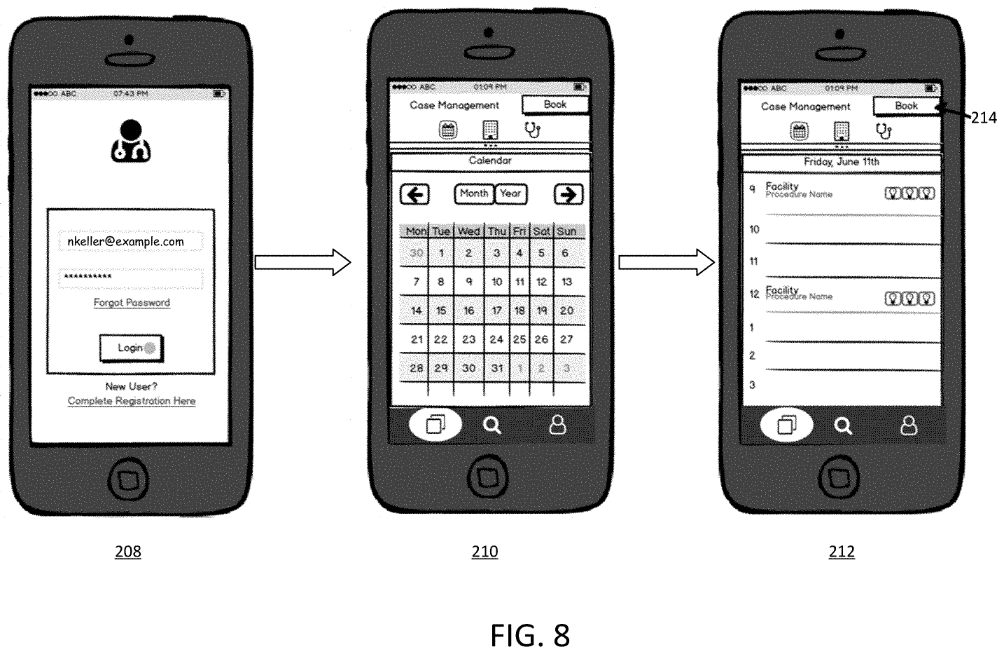

Referring now to FIG. 8, an example process diagram for case management is shown, in accordance with the present disclosure. A login screen 208 may be displayed via GUI 94. As shown, the login screen 208 may have fields for username and password. In some embodiments, the login screen 208 may also include a button for “forgot password.” The platform 32 can authenticates the username and password by accessing the user profile database 78, for example. Further, the user profile database 78 can provide user permissions as specified by the user profile.

Still referring to FIG. 8, a case management screen 210 is shown, according to some embodiments. The case management screen 210 may be displayed via GUI 94, subsequent to the login screen 208, for example. The case management screen 210 may display a monthly calendar with highlighted days where a case is scheduled. A user can select a specific day via the calendar, and GUI 94 can display a second case management screen 212. The second case management screen 212 can display an hourly day view, as well as any cases scheduled for that day. The case management screens 210, 212 can include a “book” button 214, which will be discussed in detail below.

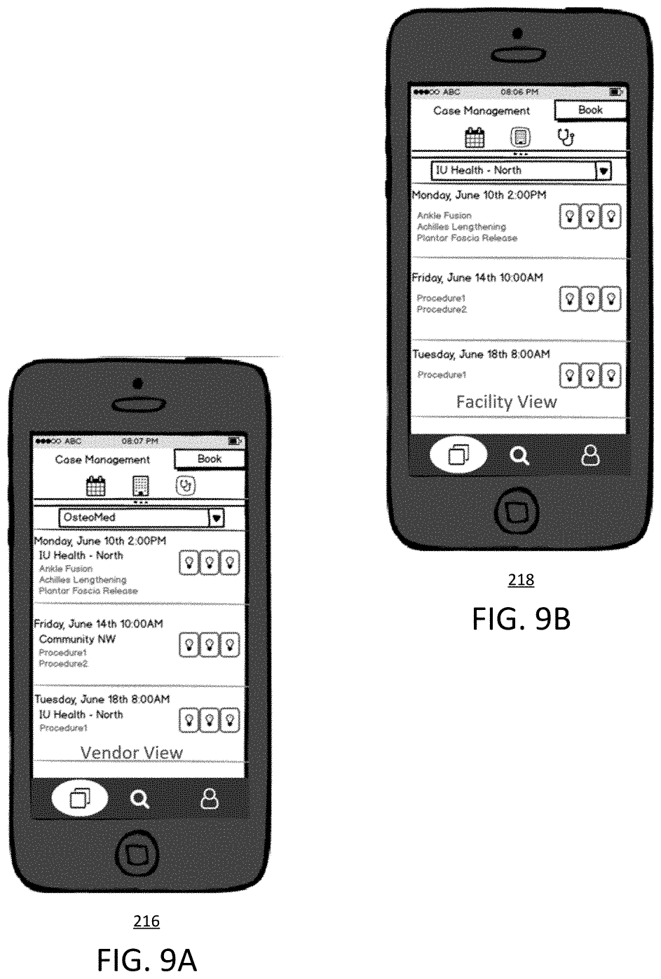

Referring to FIGS. 9A-9B, example screens corresponding to vendor and facility views are shown, in accordance with the present disclosure. In some embodiments, the case management screen 210 (e.g., corresponding to a calendar), may be the default screen upon login. As shown by FIGS. 9A-9B, additional tabs (corresponding to additional case management screens), may be included via a menu. The menu may be fixed on a ribbon within the GUI 94, such that a user can select the additional tabs at any point during case management navigation. FIG. 9A shows an example vendor view screen 216, and FIG. 9B shows an example facility view screen 218.

The vendor view screen 216 can include a list of cases scheduled for the first vendor in the displayed dropdown list. In some embodiments, a vendor dropdown list can display a list of all the user's known vendors, and allows the user to change the currently selected vendor to display cases scheduled for that vendor (e.g., ordered by date).

The facility view screen 218 can include a list of cases scheduled for the first facility in the displayed dropdown list. In some embodiments, a facility dropdown list can display a list of all the user's known facilities, and allows the user to change the currently selected facility to display cases scheduled for that facility (e.g., ordered by date).

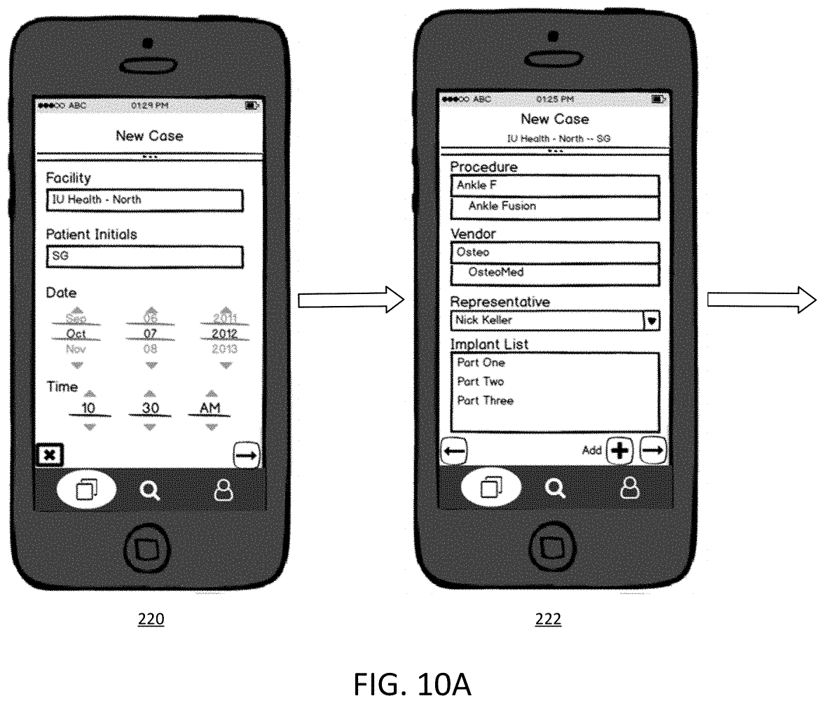

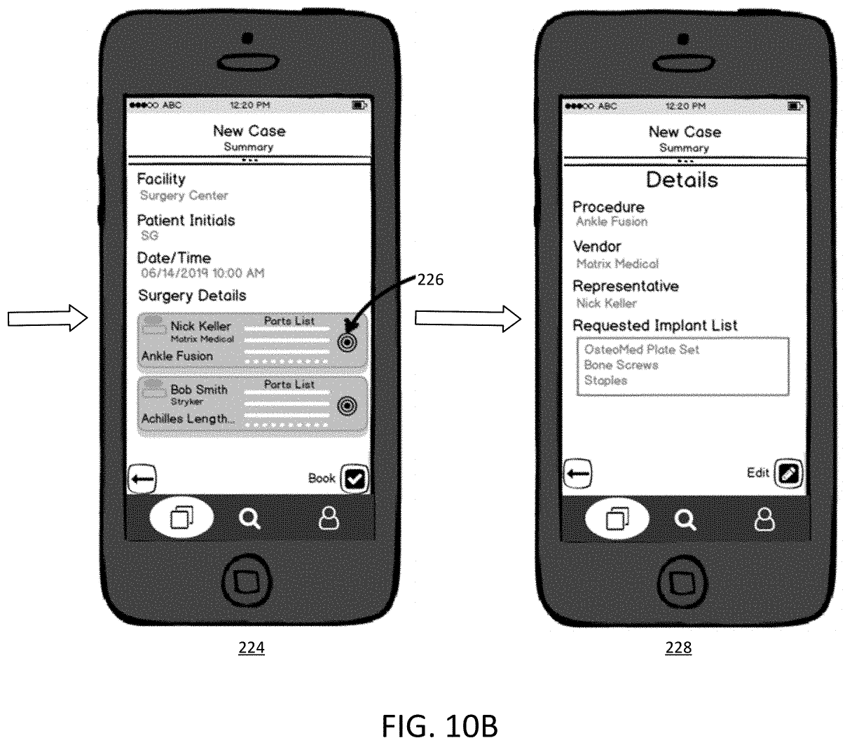

Referring particularly to FIGS. 10A-10B, an example process diagram for creating a case is shown, in accordance with the present disclosure. A new case screen 220 can be displayed via GUI 94, upon a user selecting the “book” button 214 discussed herein. The “book” button 214 may be selected from the facility view screen 218, the vendor view screen 216, or the case management screens 210, 212. The platform 32 may be configured to auto-populate certain fields and/or dropdown menus based on which screen the user selected the “book” button 214. As one example, when a user selects the book button 214 from the case management screens 210, 2012, the new case screen 220 may have the date field auto-populated based on the previously selected calendar date. As another example, when a user selects the book button 214 from the facility view screen 218, the new case screen 202 may have the facility field auto-populated based on the previously selected facility.

As shown, the new case screen 220 may have data fields for the facility, patient initials, date, and time. In some embodiments, the facility field may detect the first several letters typed, and the platform 32 may display auto-complete suggestions (e.g., sourced from the user profiles database 78). Once a user has input the relevant information into the fields provided on the new case screen 220, they may select a “next” button, and GUI 94 subsequently displays a second new case screen 222.

Still referring to FIGS. 10A-10B, the new case screen 222 is shown, according to some embodiments. The new case screen 222 may include several data fields and/or dropdown menus, as shown. In particular, the new case screen 222 can include a “procedure” field, a “vendor” field, a “representative” dropdown, and/or an “implant list.” One or more of the fields may be configured to provide auto-complete suggestions based on previous case entries associated with the present user (e.g., via the user profiles database 78). Example auto-complete suggestions are shown for the “procedure” and “vendor” fields. The implant and/or instrument list may provide auto-complete suggestions, in some embodiments. This may be especially beneficial if a surgeon uses multiple vendors, and each vendor has a particular name for their implant and/or instrument system. The surgeon will quickly be able to see which implants and/or instruments they previously used, without having to type the full implant and/or instrument names. Selecting the “add” button saves the current case information, and provides the user the option to add another case to the scheduled time slot (e.g., if the patient needs an ankle fusion and an Achilles procedure, multiple implant systems may be desired).

Once a user is finished creating the new case, they may select the “next” button. GUI 94 can subsequently display a case summary screen 224. In some embodiments, the case summary screen 224 may include all the data that was entered when the case was created (e.g., patient initials, facility, date/time, etc.). A details button 226 may prompt the GUI 94 to display a details screen 228, with information specific to the implants and/or instruments requested for the selected procedure. In some embodiments, selecting the “book” button on the case summary screen 224 can save the case information to the platform 32 (i.e., so relevant users may access the case details). Additionally, in some embodiments, selecting the “book” button can trigger a notification (e.g., a text, email, etc.), to be sent to the designated representative(s).

In some embodiments, the user may define a new case via a “clone” option. As an example, a surgeon may frequently perform the same procedure, and may have a preferred implant and/or instrument for that procedure. Instead of creating each new case from scratch, the surgeon may choose to clone a previous case. Once cloned, the surgeon can update the relevant information, such as the patient initials, and date/time. Additionally, in some embodiments, the platform 32 may define a minimum number of days required for scheduling certain procedures. As an example, if a patient-customized implant is requested for a case, the platform 32 may restrict a user from scheduling that case for a predetermined time frame (e.g., one week, one month).

In some situations, facilities may limit which vendors and/or implants are available for procedures. In some embodiments, the platform 32 may limit the implant and/or instrument options available, based on the selected facility. If a vendor or implant is not available, the dropdown menus may display those options as grayed out (i.e., unselectable). This aids the surgeon in knowing which systems are truly available for a given procedure at a specific facility. The facilities may update their available systems/implants via the platform 32, which can subsequently store this information.

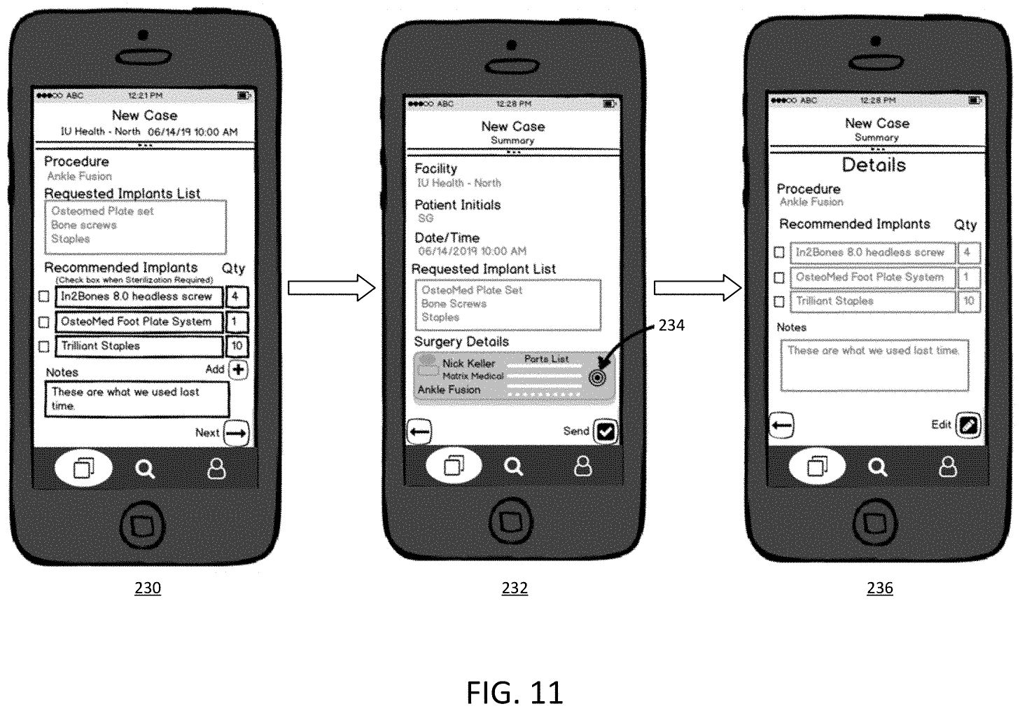

Referring now to FIG. 11, an example process diagram for creating a case is shown, in accordance with the present disclosure. In some embodiments, a representative may receive a notification of a case assignment, and may access the platform 32 to view the case details. When the representative views the new case, the GUI 94 may display the new case screen 230. As shown, the new case screen can include details about the case, as well as the requested implants and/or instruments list. The representative may review the implants and/or instruments list and input recommended implants into the “recommended implants” fields. There may be minimal differences between the requested implants list and the recommended implants list, however, this bridge between the surgeon and representative allows the surgeon to make a general request (e.g., “staples”), and the representative can refine the request if needed/desired (e.g., “Trilliant staples”). Further, the representative can accurately determine the quantities for each element within the implant and/or instruments list. As discussed herein, various fields within the new case screen 230 may be configured with auto-complete, such that the representative does not need to fully type implant and/or instrument names that were used for previous cases (the platform 32 can recognize terms and auto-complete the field).

Notably, the new case screen 230 can include a checkbox for each implant and/or instrument within the list, which may be checked if the specific implant and/or instrument requires sterilization. Further, each implant may have a specified quantity, which can be edited by the representative. Selecting the “add” button can add a new row of inputs for implants and/or instruments. In some situations, a representative may want to add notes for the specific case. Accordingly, a “notes” field can be provided via the new case screen 230. Once the user (e.g., representative) is finished editing the fields within the new case screen 230, they can select the “next” button.

Still referring to FIG. 11, a case summary screen 232 is shown, according to some embodiments. The GUI 94 can display the case summary screen 232 subsequent to the selection of the “next” button. As shown, the case summary screen 232 can include relevant fields and data entered for the particular case. The details button 234 can be selected to view the implant and/or instrument information provided by the representative via the new case screen 230. FIG. 11 includes an example case details screen 236, according to some embodiments. The case summary screen 232 can include a “send” button. In response to selection of the send button, the platform 32 can be configured to save the recommended implants and/or instrument list (e.g., to the procedure data database 76). Further, a notification can be sent to the surgeon (and/or the surgeon's assistant) via the platform 32, text, and/or email. Accordingly, the surgeon can see (in real-time) when the representative has submitted recommended implants and/or instruments for a case.

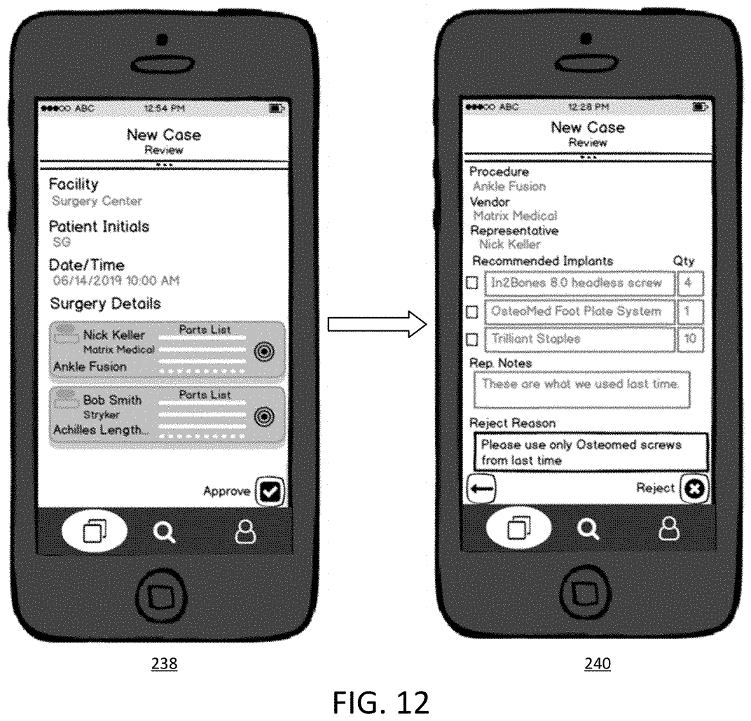

Referring particularly to FIG. 12, an example process diagram for reviewing a case is shown, in accordance with the present disclosure. GUI 94 can display a case review screen 238, according to some embodiments. The surgeon may access the case review screen 238 upon receiving a notification regarding the submission of a recommended implants and/or instruments list, or the surgeon may review as desired. The surgeon can review the recommended implants and/or instruments list by selecting the individual procedure. Upon selection of a procedure, a case details screen 240 can be displayed by GUI 94. The surgeon can reject the recommended implants list via the case details screen 240, if desired. A field for “reject reason” can be filled in with comments from the surgeon. As shown, for example, the surgeon may object to the use of a particular screw type, and can provide that feedback as part of rejecting the recommended implants. Alternatively, the surgeon can approve the recommended implants list via the case review screen 238. Selecting the “approve” button can save the case information to the platform 32, and in particular, can save the status as “approved.” In some embodiments, the representative may receive a notifications from the platform 32 when the case has been approved by the surgeon. Accordingly, the representative can see (in real-time) when the surgeon has approved or rejected the recommended implants and/or instruments.

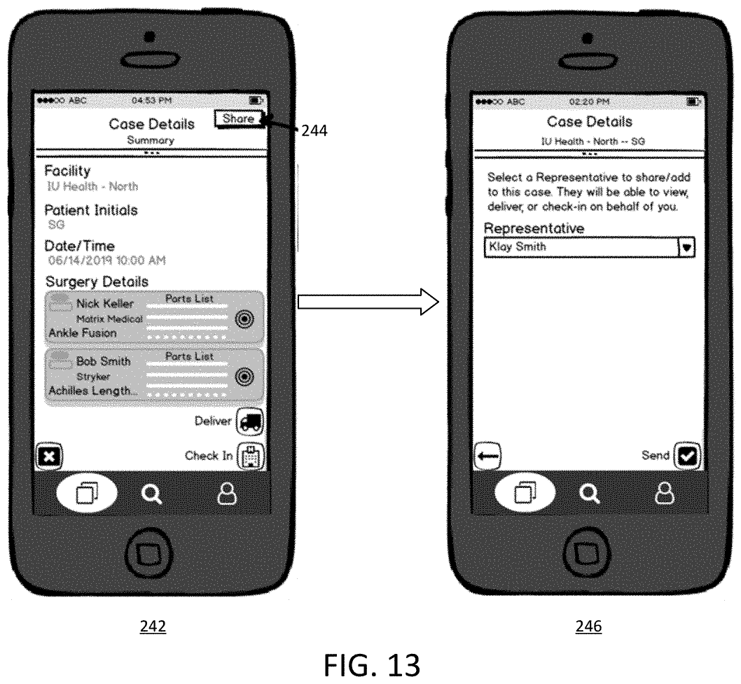

Referring particularly to FIG. 13, an example process diagram for sharing a case is shown, in accordance with the present disclosure. A case details screen 242 can be displayed via GUI 94, according to some embodiments. The case details screen 242 can include a “share” button 244. Upon selection of the share button 244, GUI 94 can display a share screen 246. In some embodiments, the share screen 246 can include a dropdown menu or text field for selecting a representative to share/add to the specific case. Notably, sharing the case can enable the selected representative to deliver the implant(s) and/or check-in to the facility on behalf of the representative assigned to the case. The platform 32 may provide suggested representatives to share the case with, based on the user's information (e.g., from user profiles database 78). In some embodiments, the platform 32 may provide suggested representatives based on the known manufacturer 40 associated with the rep 34. The share screen 246 can include a “send” button that, upon selection, prompts the platform 32 to send a notification to the selected representative (e.g., indicating that they have been added to a case). The selected representative may subsequently have access, via the platform 32, to the case information.

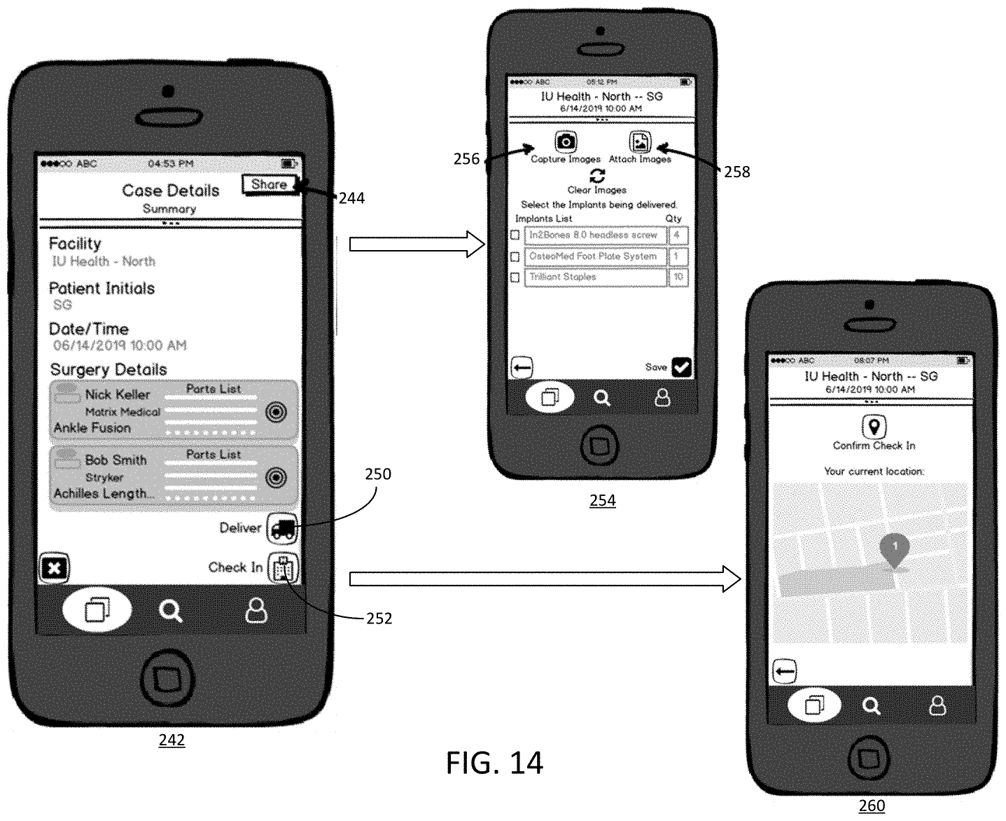

Referring particularly to FIG. 14, an example process diagram for tracking delivery and check in is shown, in accordance with the present disclosure. Again, a case details screen 242 can be displayed via GUI 94, according to some embodiments. As shown, the case details screen 242 can have a deliver button 250 and a check in button 252.

Upon selecting the deliver button 250, the GUI 94 can display a delivery screen 254. As an example, the representative may select the deliver button 250 when they arrive to the facility to drop off the implant(s) and/or instruments. The delivery screen 254 can include the implants and/or instruments list, as well as a capture images button 256 and an attach images button 258. In some embodiments, the capture images button 256 can open the camera application corresponding to the smartphone. One or more pictures can be taken of the implant(s) and/or instruments, upon delivery, via the camera application.

In some embodiments, the attach images button 258 can open the photo gallery corresponding to the smartphone, and the representative can select the implant and/or instrument image(s) to upload to the platform 32. The uploaded image(s) can be made accessible to everyone assigned to the case (via the platform 32) upon selection of the “save” button. Selection of the save button can also set the case status within the platform 32 to “delivered.” In some embodiments, in response to the case status being updated to “delivered,” the platform 32 may generate and send a notification to the surgeon (e.g., that the delivery is complete).

Still referring to FIG. 14, the case details screen 242 is shown to include the check in button 252. Upon selecting the check in button 252, the GUI 94 can display a check in screen 260. As an example, the representative may select the check in button 252 when they arrive to the facility the day of the scheduled case/procedure. The check in screen 260 can include a “confirm check in” button, as well as a visual map of the representative's current location at the facility. The platform 32 may access the GPS location coordinates (i.e., geo-spatial positioning data) of the user's device as part of the check in process. Accordingly, the GUI 94 may display a message to the user stating that their current location will be sent to the surgeon. Upon check in, the case status can be updated within the platform 32 to “check in complete.” The surgeon may receive a notification that the representative has completed check in.

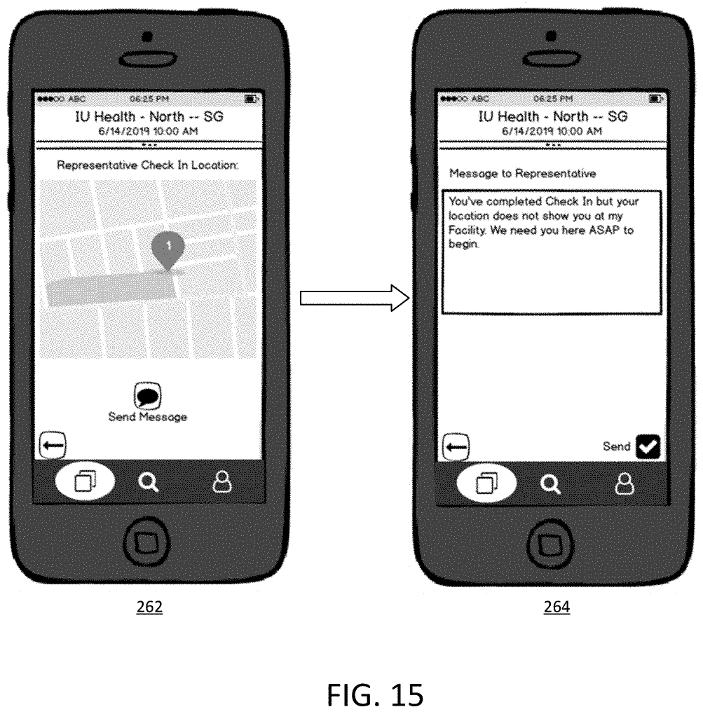

Referring particularly to FIG. 15, an example process diagram for tracking a representative is shown, in accordance with the present disclosure. In some embodiments, the surgeon may be able to view and track the representative's physical location within the facility, subsequent to the representative completing the check in process. Alternatively, the GUI 94 may display the last known GPS location corresponding to the representative's device.

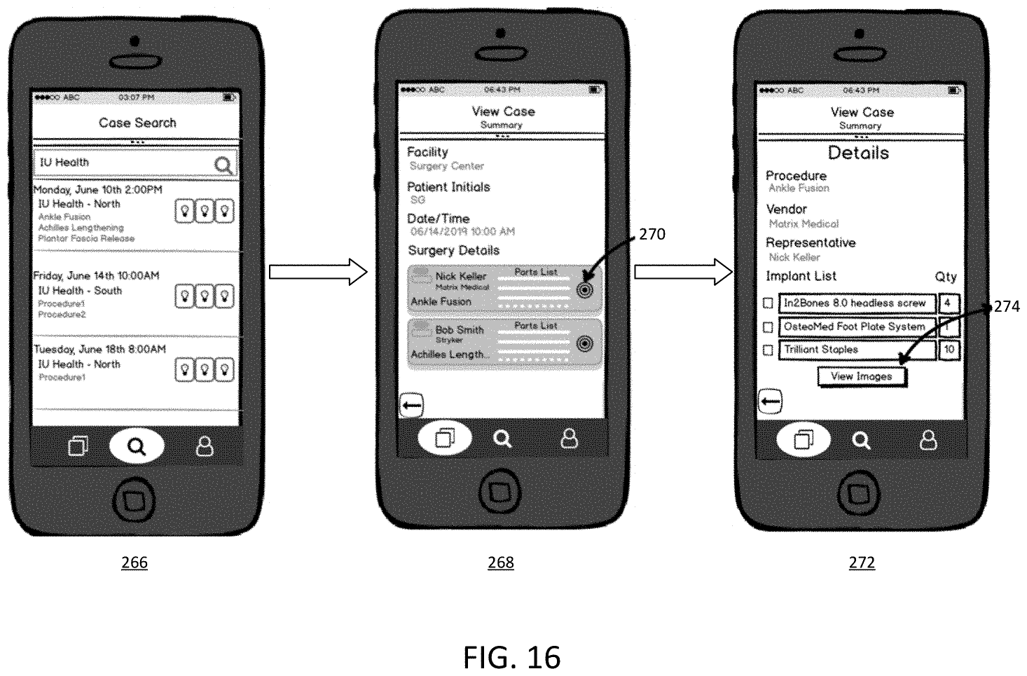

Referring particularly to FIG. 16, an example process diagram for viewing cases is shown, in accordance with the present disclosure. As shown, the GUI 94 may display a case search screen 266, according to some embodiments. The user may select a specific case to view additional details. Subsequently to selecting a case, the GUI 94 can display a case details screen 268. A user can select a procedure via a details button 270, to view additional details corresponding to the implant(s) and/or instruments. The GUI 94 may display a second case details screen 272, according to some embodiments. The second case details screen 272 can include a “view images” button 274. In some situations, the surgeon may wish to view the images of the delivered implant(s) and/or instruments, prior to the start of the procedure (e.g., to confirm that the delivery has everything they are expecting). Selecting the view images button 274 can prompt the GUI 94 to display the stored images corresponding to the delivery of the case implant(s) and/or instruments. Advantageously, this can reassure the surgeon that everything has been successfully delivered. Similarly, the charge nurse can confirm precisely what has been delivered. In some embodiments, the nurse may access the platform 32 and receive notifications corresponding to their cases (e.g., when something has been delivered, when pictures have been added to the platform 32).

In some embodiments, the platform 32 can provide a screen configured for facility personnel to confirm the delivery. As an example, facility personnel who receive the delivered implant(s) and/or instruments can verify that what the representative is delivering matches what is expected and stated as being “delivered.” In some embodiments, for example, the facility personnel can take photos of the delivered items, and the photos can be uploaded to the platform 32 (similarly to the photos that the representative may take when delivering the implant(s) and/or instruments). Further, in some embodiments, the facility personnel may sign off on the delivery, and indicate temporary possession of the implant(s) and/or instruments. Advantageously, if the surgeon is looking for the implant(s) and/or instruments, they can access the platform 32 to determine who received the delivery at the facility.

As mentioned above, the platform 32 provides a way for the surgeon (and/or other users with permission) to track when the representative has arrived at the facility. In addition to tracking general arrival, the platform 32 can access the GPS software native to the smartphone while the representative is at the facility. Accordingly, the surgeon can locate the representative based on their respective real-time locations. Advantageously, this can ensure that the representative is present when needed.

In some embodiments, the platform 32 can provide a tracking system for the delivered implant(s) and/or instruments within the facility. All facilities have their own organizational plan for where sterile instruments and implants are stored. Generally, facilities have a central room with shelves of sterile-wrapped sets. However, each facility uses their own methodology for organizing the room and sterile sets. The platform 32 can be configured to interface with a radio-frequency identification (RFID) tag using native smartphone hardware (e.g., a near-field communication application and related hardware), according to some embodiments. An RFID tag can be placed on the outside wrapping of each sterilized implant and/or instrument set. Accordingly, a user can enable a “locate” function within the platform 32 (via GUI 94), and the platform 32 can aid the user in tracking and locating the sterilized set corresponding to the case.

In some embodiments, the RFID tag can also enable a user to view the contents of the sterilized set. As an example, a user can detect the RFID tag using their smartphone, and the platform 32 (via GUI 94) can ask the user if they would like to view the contents of the set. If a user wants to view the contents, GUI 94 can display the uploaded images of the implant(s) and/or instruments. Advantageously, this can prevent the sterilized sets from being initially misidentified (e.g., when being delivered to an operating room) due to misinterpretation of the physical label on the sterilized packing. In some embodiments, the sets may be digitally “assigned” to a specific operating room via the platform 32. The platform 32 may generate an alert if the set is taken into the “wrong” operating room. Similarly, the platform 32 may generate an alert/notification when the set is taken into the correct operating room.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Finally, it is expressly contemplated that any of the processes or steps described herein may be combined, eliminated, or reordered. Accordingly, this description is meant to be taken only by way of example, and not to otherwise limit the scope of this invention.

What is claimed is:

1. A system, comprising one or more hardware computing devices including a processor and memory storing program instructions that, when executed by the processor, cause the one or more hardware computing devices to provide, to a plurality of user computing devices in electronic communication with the one or more hardware computing devices over a network, a medical device coordination platform that is configured to:
receive first user input data from a first device of a plurality of user devices, the first device associated with a medical provider;
receive second user input data from a second device of the plurality of user devices, the second device associated with a representative of a medical device provider; and
receive third user input data from a third device of the plurality of user devices, the third device associated with a medical facility;
wherein at least one of the first, second, and third user input data corresponds to a preparation status, the medical device coordination platform configured to generate and send a notification to at least one of the plurality of user devices in response to the preparation status corresponding to a predetermined status.

2. The system of claim 1, wherein the predetermined status indicates delivery of a medical device to the medical facility, the medical device coordination platform configured to generate and send the notification to the first device of the plurality of user devices.

3. The system of claim 2, wherein the medical device coordination platform is configured to display, via the first device, at least one image of the medical device upon delivery to the medical facility.

4. The system of claim 1, wherein the predetermined status indicates check-in of the representative to the medical facility, the medical device coordination platform configured to generate and send the notification to at least one of the first device and the second device.

5. The system of claim 4, wherein the medical device coordination platform is configured to receive, in response to the predetermined status indicating check-in of the representative, geo-spatial positioning data from the second device, the geo-spatial positioning data accessible via the plurality of user devices.

6. The system of claim 1, wherein the predetermined status indicates a medical device request from the medical provider, the medical device coordination platform configured to generate and send the notification to the second device of the plurality of user devices, and
wherein the medical device coordination platform is configured to receive, in response to the medical device request, a medical device recommendation from the second device.

7. The system of claim 6, wherein the medical device coordination platform is configured to generate and send a notification to the first device in response to receiving the medical device recommendation, and
wherein the medical device coordination platform is configured to display, via the first device, the medical device recommendation and a selectable reject button.

8. The system of claim 1, wherein the predetermined status indicates scheduling of a medical procedure, the medical device coordination platform configured to generate and send the notification to the third device of the plurality of user devices.

9. The system of claim 1, wherein the second user input data corresponds to contact information for a second representative of the medical device provider, and
wherein the medical device coordination platform is configured to generate and send an access invitation to a known device corresponding to the second representative.

10. The system of claim 1, wherein the medical device coordination platform is configured to open and access a camera application associated with the second user device, in response to a user input.

11. The system of claim 1, wherein the medical device coordination platform is configured to open and operate a camera application associated with the second user device, in response to a user input.

12. The system of claim 1, wherein the medical device coordination platform is configured to open and upload images from a photo gallery application associated with the second user device, in response to a user input.

13. The system of claim 1, wherein the medical device coordination platform is configured to access and communicate data with a near-field communication application associated with at least one of the plurality of user devices.

14. The system of claim 13, wherein the medical device coordination platform is configured to receive data from radio-frequency identification (RFID) tags, and
wherein the medical device coordination platform is further configured to display medical device information based on the received data from an RFID tag.

15. A method for tracking medical procedure data among entities, the method comprising:
receiving and storing a new case entry and corresponding new case data;
providing a new case notification to at least a facility device and a representative device;
receiving a request for a medical device and storing the request;
communicating the request to the representative device; and
receiving a recommended medical device associated with the request and storing the recommended medical device.

16. The method of claim 15, further comprising:
providing a notification to a physician device in response to receiving the recommended medical device; and
receiving and storing a user input of acceptance or rejection from the physician device.

17. The method of claim 15, further comprising:
receiving a check in alert from the representative device; and
providing a notification to a physician device in response to the check in alert.

18. The method of claim 17, further comprising:
prompting an upload of at least one image of a delivered medical device; and
uploading and storing the at least one image.

19. The method of claim 15, wherein the new case data includes a facility and the request for the medical device corresponds to approved medical devices at the facility.

20. The method of claim 15, wherein the recommended medical device includes a sterilization indicator for each medical device component.
